(19) United States
(12) Patent Application Publication
(10) Pub. No.: US 2022/0265713 A1
(43) Pub. Date: Aug. 25, 2022

(54) SPHINGOLIPIDS FOR GENERATING REGULATORY CD4+ T CELLS

(71) Applicant: Deutsches Krebsforschungszentrum, Heidelberg (DE)

(72) Inventors: Guoliang CUI, Heidelberg (DE); Sicong MA, Heidelberg (DE)

(73) Assignee: Deutsches Krebsforschungszentrum, Heidelberg (DE)

(21) Appl. No.: 17/620,565

(22) PCT Filed: Jun. 18, 2020
(86) PCT No.: PCT/EP2020/066942

§ 371 Date: Dec. 17, 2021

(30) Foreign Application Priority Data
Jun. 18, 2019 (EP) 19180971.4

(51) Int. Cl. A61K 35/17; C07C 215/10; C07K 16/28; C07K 14/55; C07K 14/495; A61P 37/06; C12N 5/0783

(52) U.S. Cl. CPC A61K 35/17; C07C 215/10; C07K 16/2809; C07K 16/2818; C07K 14/55; C07K 14/495; A61P 37/06; C12N 5/0637; C12N 2501/04

(57) ABSTRACT

The present invention relates to a substance of formula (I), whereby R₁ is an alkyl or alkenyl group having 6 to 20 carbon atoms; R₂ is H or missing, whereby O is bound via a double bond, R₃ is H or an acyl group -C(O)R₅, whereby R₅ is an alkyl or alkylene group having 1 to 10 carbon atoms, and R₄ is H or a phosphate group for use as a medicament and for use in a method of preventing or treating a subject suffering from an autoimmune disease. The present invention further relates to a method for generating regulatory T cells (Treg cells) in vitro comprising the steps of providing precursor CD4⁺T cells, cultivating the precursor CD4⁺T cells provided in step 1) in the presence of the substance as defined herein, and, optionally, isolating the generated regulatory T cells (Treg cells).

### BACKGROUND OF THE INVENTON

Regulatory T cells (Treg cells or Tregs) were initially described as CD4⁺CD25⁺T cells. Treg cells are mainly concerned with the regulation of the immune system. Due to their formation mechanism, Treg cells are divided into natural Treg cells (nTregs), which are differentiated in the thymus and are then transported into the periphery of the body, and induced/adaptive Treg cells (iTregs), which are generated in the periphery of the body. Treg cells are characterized by the expression of CD4 and an increased expression of the a chain of the interleukin-2 receptor (CD25^{high}, also termed as CD25⁺), as compared to the expression of CD25 in activated effector T cells (CD25^{low}, also termed as CD25⁻, T cells). Thus, Treg cells may be distinguished from effector T cells by the expression level of CD25. About 2 to 10% of the CD4⁺T cells express high levels of CD25 (CD25^{high}) and are Treg cells. Besides the high expression of CD25, Treg cells also have a low expression of the a chain of the interleukin-7 receptor (CD127^{low}, also termed as CD127⁻), as compared to the expression of CD127 in activated effector T cells (CD127^{high}, also termed as CD127⁺, T cells). Moreover, typically CD4⁺CD25^{high} Treg cells express the transcription factor Forkheadbox (Foxp3), which is essential for their development and suppressive capacity, as described hereafter.

The main focus of the regulation of the immune system by Treg cells is the suppression of the activation and also the suppression of expansion of auto-reactive effector T cells, i.e. CD4⁺and CD8⁺T cells and B cells, and the control of the activation of dendritic cells, macrophages and natural killer cells. Regulatory T cells play an essential role in the restriction of an immune response against foreign antigens and the maintenance of tolerance to autoantigens. In addition to maintaining tolerance and thus the associated prevention of autoimmune diseases, regulatory T cells play an important role in the suppressive control of immune responses to allergens and pathogenic microorganisms. They also contribute to the tolerance induction of the immune system against organ transplants and protect against excessive immune responses against the fetus during pregnancy. Thus, Treg cells promote or maintain tolerance to antigens, typically to autoantigens. Only CD4⁺CD25⁺T cells have high suppressive activity.

Autoimmune diseases have in common that the body reacts to a self-antigen. It is misinterpreted as an intruder marker and the carrier cells are attacked by the own immune system.

Treg cells have been shown to be defective in a wide variety of autoimmune diseases including thyroiditis, oophoritis, gastritis or inflammatory bowel diseases etc. These defects are manifested by loss of Treg cell number in inflamed tissues, defective Treg cells, reduced signaling through the interleukin-2 receptor and instability of the suppressive activity. Thereby, CD4⁺CD25⁺T cells expressing FOXP3 have been recognized to be of primary importance in the regulation of autoimmune reactions. For example, mutations in the Foxp3 gene result in non-functional Treg cells and may result in lethal polyautoimmune diseases with hyper-proliferative T cells in humans. Treg cells isolated from the peripheral blood of patients with multiple sclerosis (MS) have been found to have functional impairments. Studies in experimental autoimmune encephalomyelitis (EAE), the laboratory animal model of multiple sclerosis, revealed that increasing functional Treg cell numbers through adoptive transfer of CD4⁺CD25⁺T cells can provide some degree of protection.

Emergent autoimmunity in graft-versus-host disease has been described (Tivol et al., Blood, 2005).

There is a need in the art to provide effective treatment methods of autoimmune diseases which target CD4⁺CD25⁺T cells using a substance which provides low risk of side effects. This need stems from the fact that current treatment methods with immune-suppressive drugs also inactivate immune cells that are functioning, whereby the drugs bear the risk of harmful side effects.

The present invention discloses the usefulness of sphingolipids in the generation of Treg cells for the treatment of an autoimmune disease.

In the present invention, it has been shown that an autoimmune disease can be treated by promoting the generation of Treg cells by using a sphingolipid, whereby the Treg cells can be generated both in vitro as well as in vivo. The Treg cells generated by the method of the present invention may be introduced into a subject suffering from an autoimmune disease.

### DETAILED DESCRIPTION OF THE INVENTION

In the following, the present invention is described in detail. The features of the present invention are described in individual paragraphs. This, however, does not mean that a feature described in a paragraph stands isolated from a feature or features described in other paragraphs. Rather, a feature described in a paragraph can be combined with a feature or features described in other paragraphs.

The term “comprise/s/ing”, as used herein, is meant to include or encompass the disclosed features and further features which are not specifically mentioned. The term “comprise/es/ing” is also meant in the sense of “consist/s/ing of” the indicated features, thus not including further features except the indicated features. Thus, the subject-matter of the present invention may be characterized by additional features in addition to the features as indicated.

In a first aspect, the present invention provides a substance of formula (I)

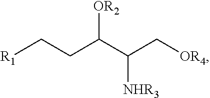

whereby

R₁ is an alkyl or alkenyl group having 6 to 20 carbon atoms;

R₂ is H or missing, whereby O is bound via a double bond,

R₃ is H or an acyl group -C(O)R₅, whereby R₅ is an alkyl or alkenyl group having 1 to 10 carbon atoms, and

R₄ is H or a phosphate group,

for use as a medicament.

In a second aspect, the present invention provides a substance of formula (I)

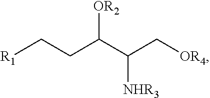

whereby

R₁ is an alkyl or alkenyl group having 6 to 20 carbon atoms;

R₂ is H or missing, whereby O is bound via a double bond,

R₃ is H or an acyl group -C(O)R₅, whereby R₅ is an alkyl or alkenyl group having 1 to 10 carbon atoms, and

R₄ is H or a phosphate group,

for use in a method of preventing or treating a subject suffering from an autoimmune disease.

In an embodiment of the above, the present invention provides the substance of formula (I), wherein the substance is sphinganine, sphinganine-1-phosphate and/or 3-keto- sphinganine, preferably wherein the substance of formula (I) is sphinganine.

In an embodiment of the above, the substance is in the erythro-form, preferably erythro- sphinganine, erythro-sphinganine-1-phosphate and/or erythro-3-keto-sphinganine, still more preferably the substance is in the D-erythro-form, still more preferably D-erythro- sphinganine, D-erythro-sphinganine-1-phosphate and/or D-erythro-3-keto-sphinganine, and most preferably D-erythro-sphinganine.

In an embodiment of the above, the substance is used in combination with an agent. In a preferred embodiment, the is agent selected from the group consisting of retinoic acid, copaxone, insulin, a molecule capable of interacting with CD3, a molecule capable of interacting with CD28, transforming growth factor β(TGF(β), interleukin-2 (IL-2), a short- chain fatty acid, a bile acid, polysaccharide A, an n3 polyunsaturated fatty acid, retinoic acid, Vitamin D (VitD), Vitamin C (VitC), a polyphenol, quercetin, resveratrol, a non- steroidal anti-inflammatory drug (NSAID), rapamycin and/or a peptide fragment from an autoreactive protein, more preferably the agent is TGFβand/or IL-2.

In a third aspect, the present invention provides a method for generating regulatory T cells (Treg cells) in vitro comprising the steps of:

1) providing precursor CD4⁺T cells,

2) cultivating the precursor CD4⁺T cells provided in step 1) in the presence of the substance of formula (I) as defined herein and, optionally

3) isolating the generated regulatory T cells (Treg cells).

In an embodiment of the above, the present invention provides the method above, further comprising the step of cultivating the precursor CD4⁺T cells in the presence of an additional compound that is capable of inducing the generation of regulatory T cells (Treg cells); preferably in the presence of a molecule capable of interacting with CD3, a molecule capable of interacting with CD28, transforming growth factor β(TGFβ), interleukin-2 (IL-2), a short-chain fatty acid, a bile acid, polysaccharide A, an n3 polyunsaturated fatty acid, retinoic acid, Vitamin D (VitD), Vitamin C (VitC), a polyphenol, quercetin, resveratrol, a non-steroidal anti-inflammatory drug (NSAID), rapamycin and/or a peptide fragment from an autoreactive protein; more preferably in the presence of TGFβand/or IL-2; still more preferably in the presence of (1) TGFβand/or IL-2; and (2) an anti- CD3 antibody and/or an anti-CD28 antibody; and/or (3) a peptide fragment; yet still more preferably in the presence of TGFβan anti-CD3 antibody and an anti-CD28 antibody or in the presence of TGFβand a peptide fragment; most preferably in the presence of TGFβ, IL-2, an anti-CD3 antibody and an anti-CD28 antibody or in the presence of TGFβ, and a peptide fragment.

In an embodiment of the above, the precursor CD4⁺T cells are nave CD4⁺T cells isolated from a subject, preferably from the spleen, lymph node or peripheral blood, or the precursor CD4⁺T cells are splenocytes or peripheral blood mononuclear cells (PBMCs) isolated from a subject, preferably isolated from intravenous blood.

In an embodiment of the above, the precursor CD4⁺T cells are isolated using flow cytometry sorting or magnetic cell sorting using cell surface markers, preferably wherein these cell surface markers are CD4⁺and CD25⁺or CD25^{high} or are CD4⁺and CD25⁺or CD25^{high} and CD12T or CD127^{low}.

In an embodiment of the above, the subject suffers from an autoimmune disease.

In an embodiment of the above, the substance in step 2) is added to a final concentration of 0.1 to 20 μM, preferably to a final concentration of 1 to 15 μM, more preferably to a final concentration of 3 to 10 μM, most preferably to a final concentration of 5 to 6.25 μM.

In an embodiment of the above, the precursor CD4⁺T cells in step 2) are cultivated for 24 to 144 hours, preferably for 24 hours to 120 hours, more preferably for 48 hours to 96 hours.

In a forth aspect, the present invention provides a regulatory T cell (Treg cell) obtainable by the method of the present invention, preferably for use as a medicament, more preferably for use in a method of preventing or treating a subject suffering from an autoimmune-disease.

In an embodiment of the present invention, the autoimmune disease is autoimmune encephalitis, autoimmune encephalomyelitis, rheumatoid arthritis, type 1 diabetes, psoriasis, autoimmune kidney disease, systemic lupus erythematosus, celiac disease, inflammatory bowel disease or graft-versus-host disease, preferably the autoimmune disease is multiple sclerosis.

In a fifth aspect, the present invention provides a kit comprising transforming growth factor beta (TGF-β) and/or interleukin-2 (IL-2) and a substance of formula (I), and optionally an additional compound that is capable of inducing the generation of regulatory T cells (Treg cells), preferably a molecule capable of interacting with CD3, a molecule capable of interacting with CD28, a short-chain fatty acid, a bile acid, polysaccharide A, an n3 polyunsaturated fatty acid, retinoic acid, Vitamin D (VitD), Vitamin C (VitC), a polyphenol, quercetin, resveratrol, a non-steroidal anti-inflammatory drug (NSAID), rapamycin and/or a peptide fragment from an autoreactive protein; preferably the kit comprises (1) TGFβ. and/or IL-2; and (2) an anti-CD3 antibody and/or an anti-CD28 antibody; and/or (3) a peptide fragment; still more preferably the kit comprises TGFβan anti-CD3 antibody and an anti-CD28 antibody or the kit comprises TGFβand a peptide fragment; most preferably the kit comprises TGFβ, IL-2, an anti-CD3 antibody and an anti-CD28 antibody or the kit comprises TGFβ, IL-2 and a peptide fragment.

This invention results from an unexpected discovery made in a follow-up study of a previous report (Wu et al., 2019). In the published report, it was found that the protein SPTLC2 (encoded by the gene SptIc2) (Hanada et al., 2003) was required for the protective T cell responses to infections. Because T cells are known to protect against infection and cancer, it was hypothesized that SPTLC2 was also required for anti-tumor T cell function. To test this hypothesis, the SptIc2^{Flox/Flox} mice (generated by Professor Xian- Cheng Jiang, Upstate University, New York) were bred with the Cd4-Cre mice (commercially available from the Jackson Laboratory) to create the SptIc2^{Flox/Flox}Cd4-C re mice. The genes SptIc2 and Cd4 encode the proteins SPTLC2 and CD4 (CD4 is a marker protein for T cells), respectively. In the SptIc2^{Flox/Flox}Cd4-Cre mice, the gene SptIc2 was defective in the CD4 protein-expressing T cells. Indeed, genetic deficiency of SptIc2 in T cells impaired anti-tumor immunity (FIG. 1A). One unexpected observation was that a subset of T cells, called regulatory T cells (Treg cells), were reduced by the SPTLC2 deficiency. Treg cells are known to promote B16 tumor growth (Klages et al., 2010). However, the increased tumor growth was correlated with a reduction of Treg cells in the sptic2^{Flox/Flox}Cd4-Cre mice. Thus, the data support the idea that SPTLC2 enhanced Treg cell formation, regardless of the microenvironment. Because Treg cells are essential in suppressing autoimmunity, inhibition of SPTLC2 might be useful to treat autoimmune diseases.

To explore the possibility of inhibiting SPTLC2 to treat autoimmunity, a mouse strain with Treg cell-specific deficiency of SptIc2, the SptIc2^{Flox/Flox}Foxp3Cre-YFP mice, was bred by crossing the SptIc2^{FloxFlox} mice with the Foxp3Cre-YFP mice (generated by Professor Alexander Rudensky, Memorial Sloan Kettering Cancer Center, New York; YFP =yellow fluorescent protein). In this strain, the gene SptIc2 is defective in the Foxp3 protein expressing-Treg cells. Using an in vitro culture assay, it was found that SPTLC2 was required for the immunosuppressive function of Treg cells (FIG. 2). Because Treg cells play a very important role in maintaining self-tolerance to prevent the development of autoimmune diseases, such as multiple sclerosis, rheumatoid arthritis, type 1 diabetes and graft-versus-host (GvH) disease, it was determined if SPTLC2 regulates autoimmunity using the EAE mouse model for multiple sclerosis.

Sptic2^{Flox/FloxF}Cre-YFP mice developed more severe EAE compared with the wildtype control mice (FIG. 3). These results prompted us to hypothesize that supplementing metabolic product downstream of SPTLC2, such as sphinganine, enhanced Treg cell generation and ameliorated autoimmune diseases. The metabolic pathway downstream of SPTLC2 is shown in FIG. 4D. To test this hypothesis, sphinganine was added to T cells in an in vitro Treg cell generation system. The cytokine TGF-βinduced Foxp3-expressing Treg cells as reported (Chen et al., 2003). Sphinganine further increased Foxp3 protein expression (FIG. 4A). On the other hand, under the inflammatory T cell (also called the interleukin-17-producing T cell, or the Th17 cell)-differentiation condition, sphinganine reduced IL-17 production. Because Th17 cells promote and Treg cells antagonize EAE development (Park et al., 2005; McGeachy et al., 2005), it was tested if sphinganine ameliorated EAE symptoms. Treatment of EAE mice with sphinganine reduced the EAE clinical scores (FIG. 5). Collectively, our experimental results suggest that 1) sphinganine increased the immunosuppressive Treg cell and decreased the inflammatory Th17 cell both in vitro and in vivo and 2) sphinganine ameliorated the symptoms of EAE.

Thus, 1) a new method to generate Treg cells to suppress inflammation and 2) a small- molecule substance to treat autoimmune diseases have been identified.

The small-molecule substance is the substance of formula (I)

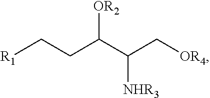

whereby

R₁ is an alkyl or alkenyl group having 6 to 20 carbon atoms;

R₂ is H or missing, whereby O is bound via a double bond, R₃ is H or an acyl group -C(O)R₅, whereby R₅ is an alkyl or alkenyl group having 1 to 10 carbon atoms, and

R₄ is H or a phosphate group.

As used herein, the term “alkyl” refers to a saturated hydrocarbon chain that may be a straight chain or branched chain, containing the indicated number of carbon atoms. The alkyl radicals in the substances of formula (I) are selected from the radicals methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl and eicosyl and the numerous different branched isomers thereof. Straight-chain and branched R₁ alkyl radicals with 10 to 16 carbon atoms are particularly preferred.

The term “alkenyl” refers to an unsaturated hydrocarbon chain which may be a straight chain or branched chain, containing the indicated number of carbon atoms. The alkenyl group may have 1, 2 or 3 unsaturated bonds. The alkenyl radicals in the substances of formula (I) are selected from the radicals ethenyl, propenyl, butenyl, pentenyl, hexenyl, heptenyl, octenyl, nonenyl, decenyl, undecenyl, dodecenyl, tridecenyl, tetradecenyl, pentadecenyl, hexadecenyl, heptadecenyl, octadecenyl, nonadecenyl and eicosenyl and the numerous different branched isomers thereof. Straight-chain and branched R₁ alkenyl radicals with 10 to 16 carbon atoms are particularly preferred.

The alkyl or alkenyl group may be unsubstituted or substituted with 1 or 2 occurrences independently selected from halogen, C₁₋₆ alkyl, C₁₋₆ haloalkyl, C₁₋₆ alkoxy, C₁₋₆ haloalkoxy, C₁₋₆ alkenyl, -OH, -NH₂ and -NH(CH₃).

The term “haloalkyl” refers to an alkyl in which one or more hydrogen atoms are replaced by halo, and includes alkyl moieties in which all hydrogens have been replaced by halo.

The term “alkoxy” refers to an -O-alkyl radical.

The term “haloalkoxy” refers to an alkoxy in which one or more hydrogen atoms are replaced by halo.

The term “halo” or “halogen” refers to a radical of fluorine, chlorine, bromine or iodine.

The most preferred substance of formula (I) is sphinganine with the formula C₁₈H₃₉NO₂, whereby in formula (I) R₁ is a C₁₃ alkyl group, R₂ is H, R₃ is H and R₄ is H.

By “a substance of formula (I)” is understood one or more, such as 2 or 3, substance(s) of formula (I).

The substance of formula (I) is described herein for use as a medicament, preferably for use in a method of preventing or treating a subject suffering from an autoimmune disease. Thereby, the substance of formula (I) works by enhancing the generation of Treg cells which are effective in ameliorating an autoimmune disease.

In an embodiment of the present invention, the substance of formula (I) is used in combination with another agent. By “agent” is meant any component which is useful or used or will be used to treat an autoimmune disease, including any component which has a general immune-suppressive activity such as glucocorticoids, such as prednisone, dexamethasone or hydrocortisone, cytostatics, alkylating agents, such as nitrogen mustards (cyclophosphamide), nitrosoureas or platinum compounds, antimetabolites, such as folic acid analogues, such as methotrexate, purine analogues, such as azathioprine or mercaptopurine, pyrimidine analogues, such as fluorouracil, or protein synthesis inhibitors, cytotoxic antibiotics, such as dactinomycin, anthracyclines, mitomycin C, bleomycin or mithramycin, antibodies, such as monoclonal antibodies, such as Muromonab-CD3, drugs acting on immunophilins, such as Ciclosporin, Tacrolimus, Sirolimus, Everolimus, Interferon, such as IFN-β, opioids, mycophenolate, or small biological agents, such as Fingolimod or Myriocin. Hence, in a preferred embodiment of the invention, the agent is an immune-suppressive agent, preferably selected from the group consisting of retinoic acid, copaxone, insulin, a molecule capable of interacting with CD3, a molecule capable of interacting with CD28, transforming growth factor β(TGFβ), interleukin-2 (IL-2), a short-chain fatty acid, a bile acid, polysaccharide A, an n3 polyunsaturated fatty acid, retinoic acid, Vitamin D (VitD), Vitamin C (VitC), a polyphenol, quercetin, resveratrol, a non-steroidal anti-inflammatory drug (NSAID), rapamycin and/or a peptide fragment from an autoreactive protein, more preferably TGFβand/or IL-2.

Moreover, “agent” includes any component which is or will be specifically used to treat a given autoimmune disease. Examples of such drugs in case of MS are Tecfidera, Gilenya, Ocrevus, Copaxone, Aubagio, Avonex, Tysabri, Rebif, Acthar, Lemtrada or drugs currently under development like S1P modulators like Ublituximab, Tysabri, Lemtrada, Arzerra, I ABT-555 or Genmab,″ Examples of such components in the case of type 1 diabetes are insulin or drugs currently under development like of sodium-glucose co- transporter (SGLT) inhibitors (Zynquista, Suglat, Forxiga, Jardiance), monoclonal antibodies (e.g. REMD-477, peplizumab), cellular therapies (e.g.VC-01, VC-02 CLBS-03 or DCVAC/Dia), interleukin receptor agonists and glucagon-like peptide 1 receptor agonists. Examples of agents for the treatment of other autoimmune diseases are known to the skilled person. Moreover, “agent” also includes such substances or components which generally promote health such as vitamins, antioxidants etc. In the context of the present invention, by “an agent” it is also understood that one or more, such as 2, 3 or 4, agent(s) can be used and/or combined.

Moreover, the present invention provides a method of generating Treg cells in vitro.

In the present invention, “regulatory T cells” or “Treg cells” are understood in the common sense, as known in the art. In particular, regulatory T-cells, as referred to herein, are T cells that have the ability to suppress pathogenic effector T cell responses or undesired effector T cell responses, more in particular to suppress pathogenic effector T cell responses against autoantigens, still more in particular to suppress pathogenic effector T- cell responses in an autoimmune disease, most in particular to ameliorate an autoimmune disease. Moreover, Treg cells, as referred to herein, may be characterized as CD4⁺CD25⁺T cells, preferably CD4⁺CD25^{high}T cells, more preferably CD4⁺CD25⁺CD127⁻T cells, still more preferably CD4⁺CD25^{high}CD127^{low}T cells. As used herein, the terms CD25⁺and CD25^{high}, the terms CD127⁺and CD127^{high}, the terms CD25⁻and CD25^{low} and the terms CD127⁻and CD127^{low} are used in alignment with the definition as known in the art (Simonetta F. et al., 2013). Furthermore, Treg cells, as referred to herein, may be characterized by the expression of the FOXP3 protein (Hori et al., 2003). Therefore, yet still more preferably, Treg cells, as referred to herein, are CD4⁺CD25^{high}CD127^{low}FOXP3⁺T cells.

Moreover, Treg cells which are generated according to the method of the present invention are preferably characterized by the production of a high amount of the FOXP3 protein (Chen et al., 2003). Moreover, Treg cells which are generated according to the method of the present invention are preferably characterized by the expression of the Programmed cell death protein 1, also known as PD-1, at high level. PD-1 is an immune checkpoint protein on the surface of cells that has a role in regulating the response of the immune system against the cells by down-regulating the immune system and promoting self-tolerance by suppressing T cell inflammatory activity (Ishida et al., 1992). This prevents autoimmune diseases. Consequently, Treg cells which are generated according to the method of the present invention have a higher immune suppressing capacity than Treg cells not treated with the substance of formula (I) (Park et al., 2016).

By the term “precursor CD4⁺T cell”, as used herein, is meant any CD4⁺T cell which can be developed into a Treg cell, preferably into a CD4⁺CD25^{high}CD127^{low}T cell, more preferably into a CD4⁺CD25^{high}CD127^{low}FOXP3⁺T cell, still more preferably into a CD4⁺CD25^{high}CD127^{low}FOXP3⁺T cell having high expression of the FOXP3 protein and high expression of the PD-1 protein, most preferably into a Treg cell of the present invention. The term “precursor CD4⁺T cell” includes non-Treg CD4⁺T cells, preferably isolated from a subject, including nave CD4⁺T cells isolated from spleen, lymph nodes, tonsils or peripheral blood mononuclear cells (PBMCs), premature CD4⁺thymocytes.

“Naïve T cells” are lymphocytes that are typically derived from the thymus and express T cell receptors. The naïve T cells have typically undergone the basic development in the bone marrow and further undergone the positive and negative processes of selection in the thymus. However, naïve T cells have not encountered their cognate antigens in the periphery. The term “differentiation” or “differentiating”, as used herein, refers to the process in which the naïve T cells are caused to further develop into differentiated T cells, being Treg cells. This is achieved by the induction of specific gene expression such that the differentiated T cell is identifiable as a particular differentiated T cell lineage, the Treg cell lineage. Naïve CD4⁺T cells are typically characterized by the expression of CD4 and are CD25^{low}or CD25⁻(CD4⁺CD25^{low} T cells or CD4⁺CD25⁻T cells).

The provision of naïve CD4⁺T cells is known in the art and can be performed by any method and from any source which allow the isolation of naïve CD4⁺T cells. The naïve CD4⁺T cells can be isolated from a body part in which they are naturally present. Thus, Treg cells can be isolated from thymus, lymph nodes including mesenteric lymph nodes, spleen or peripheral blood. Methods for isolating nave CD4⁺T cells are known in the art. A suitable method for isolating nave CD4⁺T cells includes flow cytometry sorting including fluorescence activated cell sorting (FACS) or magnetic cell sorting. Thereby, the separation procedures take into account the properties of the naïve CD4⁺T cells, e.g. CD4⁺, CD25⁻, as previously described (Berod et al., 2014).

Naïve CD4⁺T cells are then used to differentiate into Treg cells. According to the invention, differentiation is achieved by cultivating the nave CD4⁺T cells in the presence of the substance of formula (I).

In an alternative embodiment of the method of the present invention, as the precursor CD4+T cells isolated from lymph nodes, spleens or peripheral blood mononuclear cells (PBMCs) are provided. The provision of said cells is known in the art (Goyvaerts et al., 2012; Blackley et al., 2007; Bezie S. et al., 2018). Said cells can be isolated from a body part of a subject including a body fluid or body tissue. A body fluid may be, for example blood such as peripheral blood, preferably intravenous blood, or whole blood. A body tissue may be spleen, splenic tissue, or tonsils. The isolated precursor CD4+T cells such as naïve CD4⁺T cells, PBMCs or splenocytes are cultivated with a substance of formula (I). Cultivation is conducted in a suitable medium. A suitable medium is a medium allowing the generation of the Treg cells generated according to the method of the present invention. The medium is preferably a liquid medium and may be a physiologically acceptable solution, a cell culture medium or a nutrient medium. It may be with or without albumin and/or serum components. In particularly preferred embodiments, the medium is RPMI 1640 supplemented with 10% fetal calf serum, penicillin/streptomycin antibiotics and non-essential amino acids. The medium may or should be physiologically acceptable, in order to allow a course of the cultivation process of the Treg cells that comes as close as possible to the conditions present in vivo and/or in order to allow the Treg cells generated according to the method of the present invention to be infused into a subject.

The medium may contain an additional compound that is capable of inducing the production of Treg cells in general. By “an additional compound that is capable of inducing the production of Treg cells” or similar wording is meant any compound that is known or will be developed as being useful or used for promoting the generation of Treg cells which are characterized by having the ability to suppress pathogenic effector T cell responses or undesired effector T cell responses, preferably by CD4⁺CD25^{high}, more preferably CD4⁺CD25^{high}CD127^{low} and still more preferably CD4⁺CD25^{high}CD127^{low}FOXP3⁺. Thus, the medium may contain as additional compound a compound that is capable of inducing the production of Treg cells from the precursor CD4+T cells, such as a compound that is capable of causing differentiation of nave CD4⁺T cells, splenocytes or PBMCs into Treg cells. This includes a molecule capable of interacting with CD3, preferably an anti-CD3 antibody, a molecule capable of interacting with CD28, preferably an anti-CD28 antibody, TGFβ, IL-2, a short-chain fatty acid, e.g. C₁₋₆ a bile acid, e.g. isoallo lithocholic acid (Hang et al., 2018), polysaccharide A, an n3 polyunsaturated fatty acid, e.g. C₁₈₋₂₂, retinoic acid, VitD, VitC, a polyphenol EGCG (Wong et al., 2011), quercetin, resveratrol, NSAIDS, e.g. Aspirin (Javeed et al., 2009) and/or rapamycin. By “an additional compound” is understood “one or more, such as 2, 3 or 4, additional compound(s)”.

Moreover, the medium, as referred to above, may contain as additional compound a peptide fragment from an autoreactive protein (herein also termed “peptide fragment”, i.e. a self protein of a subject against which the subject can generate an auto-immune response. Thereby, the peptide fragment comprises or consists of a sequence or epitope of this protein against which effector T cells of a subject are directed. Thus, the protein and consequently the peptide fragment thereof to be added to the medium depend on the disease to be treated in a subject. For example, in a subject having type 1 diabetes, the effector T cells are insulin specific. Consequently, if the Treg cells are generated to be administered to a subject with type 1 diabetes, an insulin peptide may be added to the medium. Further examples are myelin basic protein (MBP) for MS, myelin oligodendrocyte glycoprotein (MOG) for MS, myelin associated glycoprotein (MAG) for MS or proteolipid protein (PLP) for MS. By “a peptide fragment” is understood “one or more, such as 2, 3 or 4, peptide fragment(s)”.

Preferably, in addition to the substance of formula (I) the medium contains TGFβ and/or IL-2, more preferably the medium contains (1) TGFβ and/or IL-2; (2) a molecule capable of interacting with CD3 such as an anti-CD3 antibody and/or a molecule capable of interacting with CD28 such as an anti-CD28 antibody; and/or (3) a peptide fragment, as defined above. Still more preferably the medium contains (1) TGFβ and/or IL-2; and (2) a molecule capable of interacting with CD3 such as an anti-CD3 antibody and a molecule capable of interacting with CD28 such as an anti-CD28 antibody; or (3) a peptide fragment, as defined above. Further, still more preferably the medium contains TGFβ IL- 2, a molecule capable of interacting with CD3 such as an anti-CD3 antibody and a molecule capable of interacting with CD28 such as an anti-CD28 antibody or the medium contains TGFβ, IL-2 and a peptide fragment, as defined above. Yet still more preferably, the medium contains TGFβ, an anti-CD3 antibody and an anti-CD28 antibody or the medium contains TGFβ, and a peptide fragment, as defined above. Most preferably, the medium contains TGFβ, IL-2, an anti-CD3 antibody and an anti-CD28 antibody or the medium contains TGFβ, IL-2 and a peptide fragment, as defined above.

Depending on the way of generating the Treg cells of the present invention, the medium may comprise the additional compound in a differentiating manner. Medium comprising nave CD4⁺T cells to be differentiated into Treg cells generated according to the method of the present invention preferably contains, in addition to the substance of formula (I), TGFβ and/or IL-2 and optionally a molecule capable of interacting with CD3 such as an anti-CD3 antibody and/or a molecule capable of interacting with CD28 such as an anti- CD28 antibody and more preferably TGFβ, IL-2, an anti-CD3 antibody and an anti-CD28 antibody. Medium comprising splenocytes or PBMCs preferably contains, in addition to the substance of formula (I), TGFβ and/or IL-2 and optionally a peptide fragment, as defined above, and optionally a molecule capable of interacting with CD3 such as an anti- CD3 antibody and/or a molecule capable of interacting with CD28 such as an anti-CD28 antibody and more preferably TGFβ, IL-2, a peptide fragment, as defined above, an anti- CD3 antibody and an anti-CD28 antibody.

The medium may also contain additives, which are usual in a cell culture, cell therapy and/or Treg cell cultivation. Examples of these are antibiotics, amino acid supplements, vitamin supplements and/or trace element supplements.

The concentration of precursor CD4+T cells in the medium is adapted to the total volume and preferably is 1 to 5 ×10⁶ cells per ml.

The cultivation period can be easily determined by a skilled person. Preferably, the precursor CD4+T cells are cultivated for 24 to 144 hours, preferably for 24 hours to 120 hours, more preferably for 48 hours to 96 hours, optionally in the presence of an additional compound, as defined above.

It is not necessary that the substance of formula (I) is added to the cultivation step from the beginning of cultivation. Instead, it may be added to the medium, after the precursor CD4+T cells were already cultivated in the medium for a period of time in the presence of an additional compound, as defined above. For example, cultivation of the precursor

CD4+T cells such as the nave CD4⁺T cells, splenocytes or PBMCs may occur in the presence of TGF-β/ IL-2 / a molecule capable of interacting with CD3 / a molecule capable of interacting with CD28 / a peptide fragment, as defined above, for a period of time of 12 to 72 hours, preferably 24 to 48 hours, whereby thereafter, the substance of formula (I) is added and cultivation proceeds for another period of time of 12 to 72 hours, preferably from 24 to 48, altogether from 24 to 144 hours, preferably for 24 hours to 120 hours, more preferably for 48 hours to 96 hours.

The substance of formula (I) is added to the medium to a final concentration of 0.1 to 20 μM, preferably 1 to 15 μM, more preferably 3 to 10 μM, most preferably 5 to 6.25 μM.

Cultivation takes place at a temperature which is suitable to generate Treg cells, preferably from 25 to 37° C., most preferably 37° C.

After cultivation, the medium comprising the Treg cells generated according to the method of the present invention may be used as such for purposes as needed or they may be isolated and, thus, purified to exclude other cells or components of the medium. Isolation may be performed by methods known in the art, for example to FACS-sort the cells with the markers CD4⁺CD25^{high}CD127^{low} (Bezie S. et al., 2018).

It has been shown in the present invention that by the method of the present invention using the substance of formula (I) Treg cells are generated, while the production of inflammatory IL-17 producing T cells (Th17 cells) is suppressed. This suppression of the production of Th17 cells is also obtained in the presence of TGFβ, although TGFβ induces the production of Th17 cells. Thus, the advantage of using the substance of formula (I) is, in addition to the generation of highly suppressive Treg cells capable of ameliorating an autoimmune reaction, the production of low amounts of Th17 cells. Consequently, by the administration of the substance of formula (I) to a subject, the production of Th17 cells is reduced versus the non-use of the substance of formula (I), resulting in lack or reduction of inflammatory reactions due to Th17 cells. By the administration of the Treg cells generated according to the method of the present invention to a subject, impurities of Th17 cells in the medium are low, also resulting in lack or reduction of inflammatory reactions due to Th17 cells in the subject to that the Treg cells generated according to the method of the present invention are administered. In the method of the present invention, the production of Th17 cells is greatly reduced in the presence of a substance of formula (I) versus the non-use of a substance of formula (I). Thus, in the presence of a substance of formula (I) 1 to 20%, preferably 2 to 15%, more preferably 4 to 10%, still more preferably 5 to 8% and most preferably about 6.1% of the total Foxp3 expressing CD4⁺cells (such as CD4⁺CD25^{high}CD127^{low}FOXP3⁺) present in the cultivation medium obtained in step 2) of the method of the present invention are Th17 cells. Thereby, the term “about” means ±0.5 to 10% of the indicated number, such as 0.5, 1, 2, 3, 4, 5, 6, 7, 8, 9 or 10%.

In the method of the present invention, the production of Foxp3 expressing CD4⁺cells is enhanced in the presence of a substance of formula (I) versus the non-use of a substance of formula (I). Thus, in the presence of a substance of formula (I) 31 to 90%, preferably 40 to 86%, more preferably 50 to 80%, still more preferably 60 to 75% and most preferably about 72% or 85.4% of the total CD4⁺cells present in the cultivation medium obtained in step 2) of the method of the present invention are Foxp3 expressing CD4⁺cells (such as CD4⁺CD25^{high}CD127^{low}FOXP3⁺). Thereby, the term “about” means ±0.5 to 10% of the indicated number, such as 0.5, 1, 2, 3, 4, 5, 6, 7, 8, 9 or 10%.

An autoimmune disease is a condition arising from an abnormal immune reaction against a normal part of the body. In an embodiment of the present invention, the autoimmune disease is autoimmune encephalitis, autoimmune encephalomyelitis, rheumatoid arthritis, type 1 diabetes, psoriasis, autoimmune kidney disease, systemic lupus erythematosus, celiac disease, inflammatory bowel disease, or graft-versus-host disease, preferably wherein the autoimmune disease is multiple sclerosis.

The present invention provides a medicament comprising a substance of formula (I) and a pharmaceutically acceptable carrier, which provide desirable characteristics, optionally additionally comprising an agent, as defined above. Moreover, the present invention provides a medicament comprising the Treg cells generated according to the method of the present invention and a pharmaceutically acceptable carrier, which provide desirable characteristics, optionally additionally comprising an agent, as defined above. Moreover, the present invention provides a medicament comprising a substance of formula (I), the Treg cells generated according to the method of the present invention and a pharmaceutically acceptable carrier, and optionally additionally comprising an agent, as defined above. For the production of the medicament, the substance of formula (I) and/or the Treg cells generated according to the method of the present invention and optionally the agent have to be combined in a pharmaceutical dosage form together with the a (one or more) pharmaceutically acceptable carriers.

The medicament can be manufactured for any kind of administration mode which is suitable to administer the medicament to a subject, including systemic, nasal, parenteral, vaginal, topic, rectal or oral administration. Parental administration includes subcutaneous, intracutaneous, intramuscular, intravenous or intraperitoneal administration.

The medicament can be formulated as various dosage forms including solid dosage forms for oral administration such as capsules, tablets, pills, powders, granules, liquid dosage forms for oral administration such as pharmaceutically acceptable emulsions, microemulsions, solutions, suspensions, syrups and elixirs, injectable preparations, for example, sterile injectable aqueous or oleaginous suspensions, compositions for rectal or vaginal administration, preferably suppositories, and dosage forms for dermal or transdermal administration such as ointments, pastes, creams, lotions, gels, powders, solutions, sprays, inhalants or patches. Preferably, the Treg cells generated according to the method of the present invention are formulated as a liquid dosage form, more preferably as an injectable preparation.

The specific therapeutically effective dose level for any particular subject will depend upon a variety of factors including the activity of the substance of formula (I) and/or the Treg cells generated according to the method of the present invention, dosage form, age, body weight and sex of the subject, duration of treatment and other factors known in the medical arts.

The total dose of the substance of formula (I) administered to a subject every other day for a total of 8 applications may be 1000 μg/kg body weight. The total dose of the Treg cells generated according to the method of the present invention administered to a subject in single or in multiple doses may be in amounts, for example 5×10⁶˜2.6x 10⁹ per patient, in one-time infusion as reported previously (Bluestone et al., 2015). Single dose compositions may contain such amounts or submultiples thereof to make up the daily dose.

The term “subject”, as used herein, is a mammal, such as a primate, rodent, feline, canine, or domesticated farm animal. Preferably, the subject is a human, mouse, rat, rabbit, cat, dog, cow, horse, goat or sheep.

The Treg cells generated according to the method of the present invention may be administered to a subject having an autoimmune disease. Thereby, the Treg cells generated according to the method of the present invention may be generated from autologous precursor CD4⁺T cells, such as from autologous nave CD4⁺T cells or autologous PBMCs, i.e. from a donor that is the same subject into which the Treg cells generated according to the method of the present invention are re-introduced, in order to prevent or treat an autoimmune disease. Alternatively, the Treg cells generated according to the method of the present invention may be generated from allogeneic or xenogeneic precursor CD4⁺T cells, such as from allogeneic or xenogeneic nave CD4⁺T cells or allogeneic or xenogeneic splenocytes or PBMCs, i.e. from a donor that is different (same or different species) from the subject into which the Treg cells generated according to the method of the present invention are introduced. In an embodiment, the donor may be a healthy donor, i.e. the donor does not have an autoimmune disease. Alternatively, the donor may have an autoimmune disease. It is preferred that the precursor CD4⁺T cells are autologous cells which are re-introduced into the same subject after having been treated by the method of the present invention, from which they were isolated.

Moreover, the present invention provides a method of treating or preventing an autoimmune disease, wherein the method comprises administering to a subject the substance of formula (I) in an amount sufficient to treat or prevent the autoimmune disease.

Moreover, the present invention provides a method of treating or preventing an autoimmune disease, wherein the method comprises administering to a subject the Treg cells generated according to the present invention in an amount sufficient to treat or prevent the autoimmune disease.

Moreover, the present invention provides a method of treating or preventing an autoimmune disease, the method comprising the steps of:

1) providing precursor CD4⁺T cells,

2) cultivating the precursor CD4⁺T cells provided in step 1) in the presence of the substance of formula (I),

3) optionally isolating the generated regulatory T cells (Treg cells), and

(4) returning the thus generated regulatory T cells (Treg cells) into a subject in an amount sufficient to treat or prevent the autoimmune disease.

The return can be performed in any manner suitable to introduce the Treg cells into a subject. Particularly preferred modes of return are intravenous application, intra-arterial application, intracavitary application, intrathecal application or intradermal application. Intravenous application is preferred, as this enables a direct introduction into the peripheral system and thus into the blood circulation, where the Treg cells act naturally.

### FIGURES

FIG. 1. Sptic2 deficiency in T cells increased tumor growth but decreased Treg cell formation. The Spfic2^{Flox/Flox}Cd4-Cre (FI/FI, 8 mice) and Sptic2^{+/+}Cd4-Cre (+/+, 11 mice) mice were subcutaneously implanted with 2 ×10⁵ melanoma B16 cells. The tumor sizes were measured with a caliper and calculated as length×width×width / 2. The line graph shows the tumor growth over time (A). The tumors were smashed through a 70 μM cell strainer to make single cell suspension, spun down and resuspended in 40% percoll. The 40% percoll containing tumor cells and tumor-infiltrating cells was loaded onto 80% percoll and spun at 2000 rpm for 15 min. The tumor-infiltrating leukocytes were found in the middle layer between the 40% and 80% percoll after centrifugation and collected for FACS staining and cell counting. The bar graph shows the density of FOXP3 protein- expressing Treg cells in the tumor, calculated as the number of Treg cells divided by the weight of tumors (B). Data are expressed as mean ±SEM and cumulative of 3 (A) and 2 (B) independent experiments. *, p<0.05, Student's t-test.

FIG. 2. Sptic2 deficiency in Treg cells affected the immunosuppressive function of Treg cells. YFP-positive CD4⁺Treg cells and YFP-negative CD4⁺non-Treg cells were FACS-sorted. 2 ×10⁴ non-Treg cells were cultured in 250 μl complete medium in the presence of T cell stimuli anti-CD3 and anti-CD28-coated microbeads (4 x 10⁴ microbeads per cell culture; 16 ng/ml anti-CD3 and 16 ng/ml anti-CD28. Non-Treg cells were co- cultured with the Treg cells at the indicated ratios (e.g. “8:1” means 2 ×10⁴ non-Treg cells plus 2.5 ×10³ Treg cells). The line graph shows suppression% over non-Treg:Treg cells. Data are cumulative of 3 independent experiments. Results are expressed as mean±SEM. *, p<0.05, Student's t-test.

FIG. 3. Sptic2 deficiency in Treg cells enhanced autoimmunity in the EAE mouse model. The line graph shows the EAE clinical scores over days post EAE induction. Five pairs of mice were used. Results are expressed as mean±SEM. *, p<0.05, Student's t- test.

FIG. 4. Sphinganine promoted the Treg cell in vitro generation and suppressed the inflammatory Th17 cell formation. The YFP-negative CD4⁺non-Treg naïve T cells were FACS-sorted (1 ×10⁵ cells in 250 μl complete medium) from the Foxp3Cre-YFP mice. The naive T cells were activated with the anti-CD3 and anti-CD28 for three days, in the presence or absence of cytokine TGFβ (5 ng/ml) and IL-2 (10 ng/ml) for Treg cells and TGFβ (5 ng/ml), IL-6 (20 ng/ml) and anti-IFNy (10 μg/ml) for Th17 cells. A: flow cytometry analysis of Foxp3 protein expression after induction of Treg cell formation with sphinganine (5 μM) in the presence of cytokine TGFβ (5 ng/ml) or vehicle control DMSO (no TGFβ). B: Flow cytometry analysis of IL-17 protein expression after induction of Th17 cell formation with TGFβ (5 ng/ml), IL-6 (20 ng/ml) and anti-IFNy (10 μg/ml) in the presence or absence of sphinganine (5 μM). C: Flow cytometry analysis of Foxp3 protein expression after induction of Treg cell formation in the presence of TGFβ (5 ng/ml) and L- serine (5 μM), 3-KDS (5 μM), shinganine (5 μM), dihydroceramide (50 nM), ceramide (50 nM), sphingosine (1 μM), sphingosine-1-phosphate (1 μM), shinganine-1-phosphate (1 μM), or vehicle control DMSO (TGFβ only). D: Structures of sphingolipids, as indicated, and sphingolipid biosynthetic pathway are depicted. The numbers in each FACS plot show the percentages of the indicated cell population within the total population of cells shown in the FACS plot.

FIG. 5. Sphinganine treatment ameliorated EAE development. The line graph shows the EAE clinical scores over days post EAE induction. Results are expressed as mean ±SEM. *, p<0.05, **, p<0.01, Student's t-test.

### EXAMPLES

Methods

Mice. Sptic2^{FI/FI}and Foxp3^{Cre} mice on a C57BL/6 background were provided by Professor Xian-cheng Jiang (SUNY Downstate Medical Center, New York) and Professor Alexander Rudensky (Memorial Sloan Kettering Cancer Center, New York), respectively. Cd4^{Cre} mice on a C57BL/6 background were purchased from the Jackson Laboratory. All mice were maintained in the DKFZ specific pathogen-free facility. Age- and gender-matched littermates (5-10 weeks old) were used as control mice in all experiments. All the studies were performed in accordance with DKFZ regulations after approval by the German regional council at the Regierungsprasidium Karlsruhe.

B16 melanoma implantation and tumor-infiltrating immune cell preparation. B16-F10 melanoma cells were subcutaneously injected to the Sptic2^{Flox/Flox}Cd4-Cre, Sptic2^{+/+}Cd4- Cre, Sptic2^{Flox/Flox}Foxp3-Cre-YFP and Sptic2^{+/+}Foxp3-Cre-YFP mice (2×10⁵ cells per mouse). Tumors were measured every 2-3 days using a caliper. Mice were sacrificed at the indicated time points and tumors were harvested using forceps and scissors. Tumors were smashed through 70-μm cell strainers to generate single-cell resuspension. The tumor cells were centrifuged and resuspended in 40% percoll and loaded to 80% percoll for gradient centrifugation. The immune cells were found in the middle layer between the 40% and 80% percoll after centrifugation. The immune cells were then sucked out to a new tube for flow cytometry staining.

EAE induction and monitoring. Each mouse was subcutaneously immunized with 200 μg MOG35-55 peptide emulsified in Freund's Complete Adjuvant. Pertussis toxin (400 ng per mouse) was injected intraperitoneally. Where indicated, mice were intraperitoneally injected with sphinganine (every other day from the day of immunization to the end of the experiments). EAE symptoms were scored every day using the following scoring standard: 0, no sign; 1, limp tail; 2, paraparesis (incomplete paralysis of 1 or 2 hind limbs); 3, paraplegia (complete paralysis of 2 hind limbs); 4, paraplegia with forelimb weakness or paralysis; 5, moribund state or death. For the mouse welfare reason, we sacrificed the mice if the score reached 3.

Mouse primary T cell culture. Complete medium was used for cell culture and was prepared by supplementing RPMI 1640 plain medium with 10% fetal calf serum, penicillin/streptomycin antibiotics and non-essential amino acids. For Treg and Th17 cell in vitro differentiation, naïve splenic T cells were purified from the Sptic2^{+/+}Foxp3Cre-YFP wild type mice using the flow cytometry sorter. Foxp3 is expressed in the cell nucleus and FACS staining of FOXP3 protein requires cell fixation and permeabilization, which kills cells and is not suitable for subsequent cell culture. In this Foxp3Cre-YFP mouse strain, the FOXP3 protein expression is reported by the YFP protein expression. FOXP3- expressing cells could not directly be FACS-sorted without fixing and permeabilizing cells and thus preserve cell viability. YFP-positive CD4⁺Treg cells and YFP-negative CD4⁺non- Treg cells were FACS-sorted. The naïve CD4⁺T cells (1 ×10⁵ cells in 250 μl medium) were activated with the T cell stimuli anti-CD3 and anti-CD28 for three days, in the presence or absence of cytokine TGFβ (5 ng/ml) and IL-2 (10 ng/ml) for Treg cells and TGFβ (5 ng/ml), IL-6 (20 ng/ml) and anti-IFNy (10 μg/ml) for Th17 cells. Sphinganine or other sphingolipids were added as indicated (1 or 5 μM). The Foxp3 and interleukin-17(IL-17) protein expression was examined by flow cytometry assay.

Flow Cytometry. FACS buffer (PBS with 0.5% FCS) was used to stain cell surface antigen. To stain intracellular antigens, cells were fixed with Biolegend fixation buffer (for cytokines) or eBioscience Fixation/permeabilization buffer (for transcription factors). Dead cells were excluded using the LIVE/DEAD Fixable Dead Cell Stains (Thermo Fisher Scientific). For Treg cell co-culture suppression assay, responder cells were labeled with Celltrace Violet (37° C., 20 minutes) and washed with RPMI 1640 medium with 1% FBS for 3 times. Samples were run on the LSR II and analyzed using the Flowjo software (FlowJo, LLC, BD).

Antibodies and cytokines. The antibodies were ordered from Biolegend, eBioscience and BD Biosciences for detecting the following antigens using flow cytometry: CD4 (GK1.5), IL-17 (TC11-18H10.1), and Foxp3 (FJK-16s). Anti-CD3 (17A2) and anti-CD28 (37.51) were used for cell culture.

Example 1

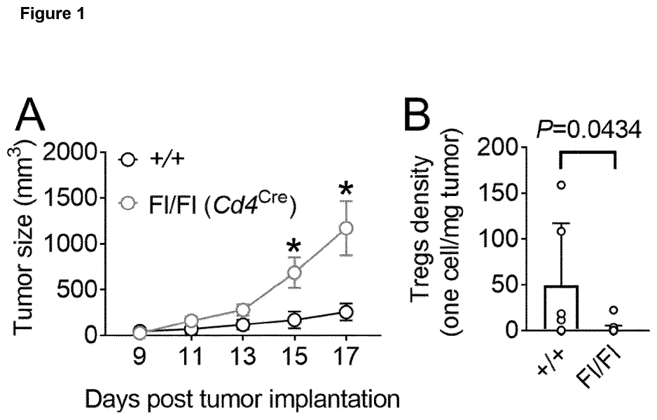

Sptic2 deficiency in T cells increased tumor growth but decreased Treg cell formation. The Sptic2^{Flox/Flox}Cd4-Cre (FI/FI, 8 mice) and Sptic2^{+/+}Cd4-Cre (+/+, 11 mice) mice were implanted with 2 ×10⁵ melanoma B16 cells. Results are shown in FIG. 1. We observed that the genetic deficiency of Sptic2 in T cells impaired anti-tumor immunity (FIG. 1A) and we further observed that a subset of T cells, called regulatory T cells (Treg cells), were reduced by the SPTLC2 deficiency (FIG. 1B).

Example 2

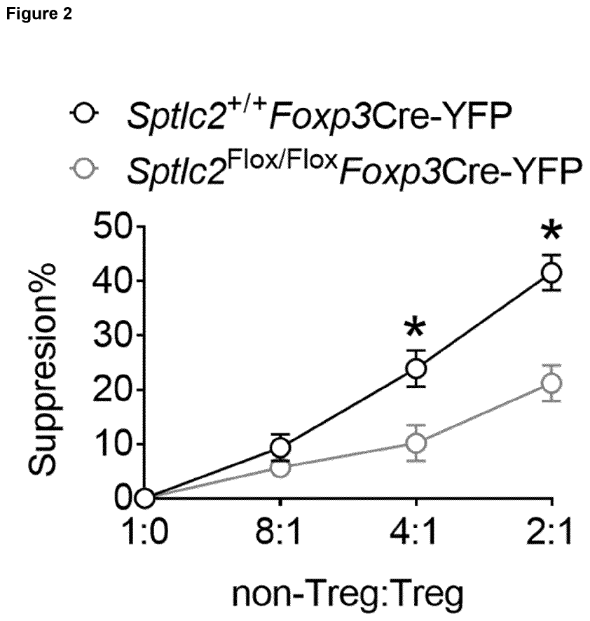

Sptic2 deficiency in Treg cells affected the immunosuppressive function of Treg cells. We purified the Treg and non-Treg cells from the Sptic2^{Flox/Flox}Foxp3Cre-YFP mice or Sptic2^{+/+}Foxp3Cre-YFP mice using the flow cytometry sorter. Foxp3 is expressed in the cell nucleus and FACS staining of FOXP3 protein requires cell fixation and permeabilization, which kills cells and is not suitable for subsequent cell culture. In this Foxp3Cre-YFP mouse strain, the FOXP3 protein expression is reported by the YFP protein expression. We could not directly FACS-sort FOXP3 xpressing cells without fixing and permeabilizing cells and thus preserve cell viability. YFP-positive CD4⁺Treg cells and YFP-negative CD4⁺non-Treg cells were FACS-sorted. The non-Treg cells were labeled with a fluorescent dye Celltrace Violet (CTV, to determine cell proliferation) and co- cultured with or without the Sptic2-deficient or -sufficient Treg cells for three days in the presence of T cell stimuli anti-CD3 and anti-CD28. The suppression % was calculated as [(T cell proliferation rate without Treg cells - T cell proliferation rate with Treg cells)/ T cell proliferation rate without Treg cells %]. We found that SPTLC2 was required for the immunosuppressive function of Treg cells. Results are shown in FIG. 2.

Example 3

Sptic2 deficiency in Treg cells enhanced autoimmunity in the EAE mouse model.

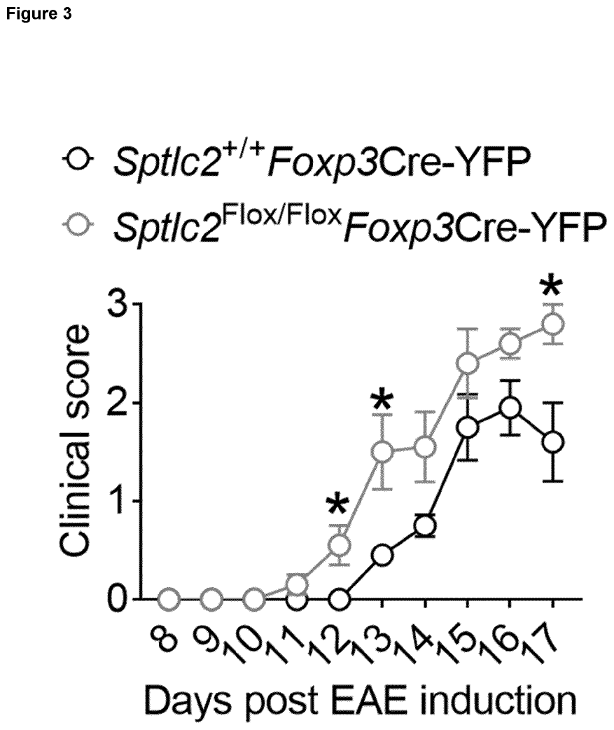

EAE was induced in the Sptic2^{Flox/Flox}Foxp3Cre-YFP mice or Sptic2^{+/+}Foxp3Cre-YFP mice. Briefly, each mouse was subcutaneously immunized with 200 μg MOG₃₅₋₅₅ peptide emulsified in Freund's Complete Adjuvant. Pertussis toxin (400 ng per mouse) was injected intraperitoneally. EAE symptoms were scored every day. Sptic2^{Flox/Flox}Foxp3Cre- YFP mice developed more severe EAE compared with the wildtype control mice. Results are shown in FIG. 3.

Example 4

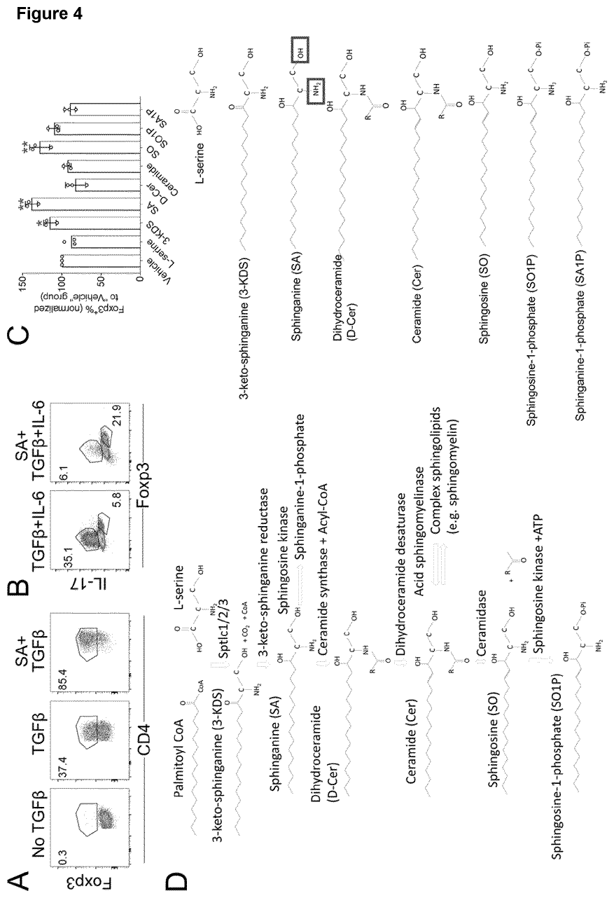

Sphinganine promoted the Treg cell in vitro generation and suppressed the inflammatory Th17 cell formation. Naïve splenic T cells were purified from the Sptic2^{+/+}Foxp3Cre-YFP wild type mice using the flow cytometry sorter. The naïve CD4⁺T cells were activated with the T cell stimuli anti-CD3 and anti-CD28 and IL-2 for three days, in the presence or absence of cytokine TGF-β(FIG. 4A, to induce Treg cell formation) or TGF-βplus interleukin-6 (IL-6) (FIG. 4B, to induce Th17 cell formation). Sphinganine (5 μM) or vehicle control DMSO was added. The Foxp3 and interleukin-17 (IL-17) protein expression was examined by flow cytometry assay. Alternatively, naïve CD4⁺T cells were cultured under the Treg cell-inducing condition (the same as to FIG. 4A) in the presence of sphinganine or other sphingolipids or vehicle control before flow cytometry analysis of Foxp3 protein expression (FIG. 4C). The structures and the sphingolipid biosynthetic pathway are depicted (FIG. 4D). The cytokine TGF-βinduced Foxp3-expressing Treg cells as reported (Chen et al., 2003). It was found that sphinganine or other sphingolipids increase Foxp3 protein expression, induce Treg cell formation and reduce IL-17 protein expression.

Example 5

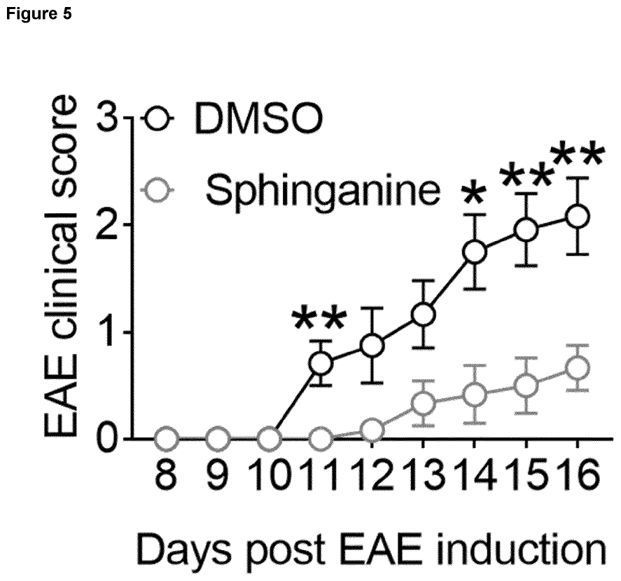

Sphinganine treatment ameliorated EAE development. C57BL/6 mice were subcutaneously immunized with 200 μg MOG₃₅₋₅₅ peptide emulsified in Freund's Complete Adjuvant to induce EAE development. Pertussis toxin (400 ng per mouse) was injected intraperitoneally. Mice were intraperitoneally injected with sphinganine (1000 μg/kg body weight, one injection every two days, from day 1 to day 15). EAE symptoms were scored every day. Results are shown in FIG. 5.

### REFERENCES

Berod L. et al., Nature Medicine 20:1327-1333, 2014.

Bezie S. et al., Frontiers in Immunology 8, Article 2014, 2018.

Blackley S. et al., Journal of Virology 81: 13325-13334, 2007.

Bluestone J. A. et al., Science Translational Medicine 7: 315ra189, 2015.

Chen W. et al., The Journal of experimental medicine 198: 1875-1886, 2003.

Goyvaerts C. et al., Gene Therapy 19: 1133-1140, 2012.

Hanada K. et al., Biochim Biophys Acta 1632:16-31, 2003.

Hang S. et al., Biorxiv doi: https://doi.org/10.1101/465344, 2018.

Hod S. et al., Science 299:1057-1061, 2003.

Ishida Y. et al., EMBO Journal 11:3887-3895 1992.

Javeed A. et al., Transplantation Immunology 20:253-260, 2009.

Klages K. et al., Cancer research 70: 7788-7799, 2010.

McGeachy M. J. et al., J Immunol. 175: 3025-3032, 2005.

Park H. et al., Nature immunology 6: 1133, 2005.

Park B. et al., Cancer Discovery 6:1366-1381 2016.

Simonetta F. et al., Frontiers in Immunology 4, Article 215, 2013.

Tivol et al., Blood. 2005 Jun. 15; 105(12): 4885-4891.

Wong C. et al., Immunology Letters 139: 7-13, 2011.

Wu J. et al., Immunity 50: 1-14, 2019.

1. A substance of formula (I) 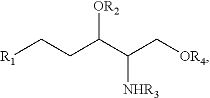
whereby
R₁ is an alkyl or alkenyl group having 6 to 20 carbon atoms;
R₂ is H or missing, whereby O is bound via a double bond,
R₃ is H or an acyl group -C(O)R₅, whereby R₅ is an alkyl or alkylene group having 1 to 10 carbon atoms, and
R₄ is H or a phosphate group,
for use as a medicament.

2. A substance of formula (I) 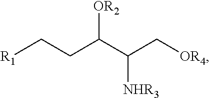
whereby
R₁ is an alkyl or alkenyl group having 6 to 20 carbon atoms;
R₂ is H or missing, whereby O is bound via a double bond,
R₃ is H or an acyl group -C(O)R₅, whereby R₅ is an alkyl or alkylene group having 1 to 10 carbon atoms, and
R₄ is H or a phosphate group,
for use in a method of preventing or treating a subject suffering from an autoimmune disease.

3. The substance for use according to claim 1 wherein the substance is sphinganine, sphinganine-1-phosphate and/or 3-keto-sphinganine, preferably wherein the substance is sphinganine.

4. The substance for use according to claim 1, wherein the substance is in the erythro-form, preferably erythro-sphinganine, erythro- sphinganine-1-phosphate and/or erythro-3-keto-sphinganine, still more preferably wherein the substance is in the D-erythro-form, still more preferably D-erythro-sphinganine, D- erythro-sphinganine-l-phosphate and/or D-erythro-3-keto-sphinganine, and most preferably D-erythro-sphinganine.

5. The substance for use according to claim 1, wherein the substance is used in combination with an agent, preferably selected from the group consisting of retinoic acid, copaxone, insulin, a molecule capable of interacting with CD3, a molecule capable of interacting with CD28, transforming growth factor β (TGFβ), interleukin-2 (IL-2), a short-chain fatty acid, a bile acid, polysaccharide A, an n3 polyunsaturated fatty acid, retinoic acid, Vitamin D (VitD), Vitamin C (VitC), a polyphenol, quercetin, resveratrol, a non-steroidal anti-inflammatory drug (NSAID), rapamycin and/or a peptide fragment from an autoreactive protein, more preferably selected from transforming growth factor β(TGFβ) and/or interleukin-2 (IL-2).

6. A method for generating regulatory T cells (Treg cells) in vitro comprising the steps of:
1) providing precursor CD4⁺T cells,
2) cultivating the precursor CD4⁺T cells provided in step 1) in the presence of a substance of formula (I) 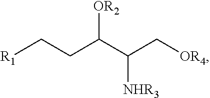
whereby
R₁ is an alkyl or alkenyl group having 6 to 20 carbon atoms;
R₂ is H or missing, whereby O is bound via a double bond,
R₃ is H or an acyl group -C(O)R₅, whereby R₅ is an alkyl or alkylene group having 1 to 10 carbon atoms, and
R₄ is H or a phosphate group, and, optionally,
3) isolating the generated regulatory T cells (Treg cells).

7. The method of claim 6, wherein the substance of formula (I) is sphinganine, sphinganine-1-phosphate and/or 3-keto-sphinganine, preferably wherein the substance is sphinganine.

8. The method according to claim 6, wherein the substance of formula (I) is in the erythro-form, preferably erythro-sphinganine, erythro- sphinganine-1-phosphate and/or erythro-3-keto-sphinganine, more preferably wherein the substance is in the D-erythro-form, still more preferably D-erythro-sphinganine, D-erythro- sphinganine-1-phosphate and/or D-erythro-3-keto-sphinganine, and most preferably D- erythro-sphinganine.

9. The method according to claim 6, further comprising the step of cultivating the precursor CD4⁺T cells in the presence of an additional compound that is capable of inducing the generation of regulatory T cells (Treg cells);
preferably in the presence of a molecule capable of interacting with CD3, a molecule capable of interacting with CD28, transforming growth factor β (TGFβ), interleukin-2 (IL- 2), a short-chain fatty acid, a bile acid, polysaccharide A, an n3 polyunsaturated fatty acid, retinoic acid, Vitamin D (VitD), Vitamin C (VitC), a polyphenol, quercetin, resveratrol, a non-steroidal anti-inflammatory drug (NSAID), rapamycin and/or a peptide fragment from an autoreactive protein;
more preferably in the presence of TGFβand/or IL-2;
still more preferably in the presence of (1) TGFβand/or IL-2; and (2) an anti-CD3 antibody and/or an anti-CD28 antibody; and/or (3) a peptide fragment;
yet still more preferably in the presence of TGFβ, an anti-CD3 antibody and an anti-CD28 antibody or in the presence of TGFβ and a peptide fragment;
most preferably in the presence of TGFβ, IL-2, an anti-CD3 antibody and an anti-CD28 antibody or in the presence of TGFβ, IL-2 and a peptide fragment.

10. The method according to claim 6, wherein the precursor CD4⁺T cells are naïve CD4⁺T cells isolated from a subject, preferably from the spleen, lymph node or peripheral blood, or wherein the precursor CD4⁺T cells are splenocytes or peripheral blood mononuclear cells (PBMCs) isolated from a subject, preferably isolated from intravenous blood.

11. The method according to claim 6, wherein the precursor CD4⁺T cells are isolated using flow cytometry sorting or magnetic cell sorting using cell surface markers, preferably wherein these cell surface markers are CD4⁺and CD25⁺or CD25^{high} or are CD4⁺and CD25⁺or CD25^{high} and CD127⁻or CD127^{low}.

12. The method according to claim 10, wherein the subject suffers from an autoimmune disease.

13. The method according to claim 6, wherein the substance in step 2) is added to a final concentration of 0.1 to 20 μM, preferably to a final concentration of 1 to 15 μM, more preferably to a final concentration of 3 to 10 μM, most preferably to a final concentration of 5 to 6.25 μM.

14. The method according to claim 6, wherein the precursor CD4⁺T cells in step 2) are cultivated for 24 to 144 hours, preferably for 24 hours to 120 hours, more preferably for 48 hours to 96 hours.

15. A regulatory T cell (Treg cell) obtainable by the method according to claim 6, preferably for use as a medicament, more preferably for use in a method of preventing or treating a subject suffering from an autoimmune-disease.

16. The substance of Formula 1 for use according to the method of claim 12, wherein the autoimmune disease is autoimmune encephalitis, autoimmune encephalomyelitis, rheumatoid arthritis, type 1 diabetes, psoriasis, autoimmune kidney disease, systemic lupus erythematosus, celiac disease, inflammatory bowel disease or graft-versus-host disease, preferably wherein the autoimmune disease is multiple sclerosis.

17. A kit comprising transforming growth factor beta (TGF-β) and/or interleukin-2 (IL-2), and a substance as defined in claim 1, and optionally an additional compound that is capable of inducing the generation of regulatory T cells (Treg cells), preferably a molecule capable of interacting with CD3, a molecule capable of interacting with CD28, a short-chain fatty acid, a bile acid, polysaccharide A, an n3 polyunsaturated fatty acid, retinoic acid, Vitamin D (VitD), Vitamin C (VitC), a polyphenol, quercetin, resveratrol, a non-steroidal anti-inflammatory drug (NSAID), rapamycin and/or a peptide fragment from an autoreactive protein;
more preferably the kit comprises (1) TGFβand/or IL-2; and (2) an anti-CD3 antibody and/or an anti-CD28 antibody; and/or (3) a peptide fragment;
still more preferably the kit comprises TGFβ, an anti-CD3 antibody and an anti-CD28 antibody or the kit comprises TGFβ and a peptide fragment;
most preferably the kit comprises TGFβ, IL-2, an anti-CD3 antibody and an anti-CD28 antibody or the kit comprises TGFβ, IL-2 and a peptide fragment.
